(19) United States
(12) Patent Application Publication
(10) Pub. No.: US 2009/0099557 A1
(43) Pub. Date: Apr. 16, 2009

(54) Methods and compositions for optimizing the outcomes of refractive laser surgery of the cornea

(76) Inventor: Olivia N. Sedarevic, New York, NY (US)

Correspondence Address: ALBERT JACOBS LLP, Suite 502, 900 Third Avenue, New York, NY, 10022, US

(21) Appl. No.: 12/316,547

(22) Filed: Dec. 12, 2008

Related U.S. Application Data
(63) Continuation-in-part of application No. 11/624,945, filed on Jan. 19, 2007.
Application No. 11/624,945 is a continuation-in-part of application No. 11/618,860, filed on Dec. 31, 2006.
Application No. 11/618,860 is a continuation-in-part of application No. PCT/US07/14018, filed on Jun. 15, 2007.
(60) Provisional application No. 60/814,097, filed on Jun. 15, 2006.

(51) Int. Cl. A61F 9/008

(52) U.S. Cl. 606/5

(57) ABSTRACT

Disclosed herein are methods and compositions for use in surgical procedures for refractive ablation of the cornea to achieve vision correction with a minimum of undesirable side effects and for a broad range of optical conditions such myopia, hyperopia, presbyopia and astigmatism. Specifically disclosed are compositions, and methods involving their use, wherein the compositions act as agents for the reversible removal of corneal epithelial layers to provide access for UV radiation in manipulation of the refractive properties of the cornea. The methods and compositions of the present invention are capable of achieving desirable results in corrective surgery not possible with current methods for exposing the corneal stroma to far-UV laser radiation.

This application is a continuation-in-part of PCT/US2007/014018, filed Jun. 15, 2007 and is a continuation-in-part of U.S. patent application Ser. No. 11/624,945, filed Jan. 19, 2007 and a continuation-in-part of U.S. application Ser. No. 11/618,860 filed Dec. 31, 2006. This application also claims priority from provisional application No. 60/814,097 filed Jun. 15, 2006. All of these applications are incorporated herein by reference in their entirety.

### FIELD OF THE INVENTION

The present invention relates generally to methods and compositions for optimization of the outcomes of far-UV laser surgery of the cornea, wherein the methods involve the use of chemical and/or pharmaceutical agents for reversible removal of the corneal epithelium in such a manner as to provide an optimally smooth, exposed corneal surface for refractive correction and rapid, tight reattachment of the epithelial layer, while simultaneously minimizing or eliminating avoidable, adverse wound healing responses implicated in undesirable side effects observed from such surgery.

### BACKGROUND OF THE INVENTION

Laser refractive surgery, using light energy from far-UV excimer lasers, has undergone a significant evolution during the last two decades, emerging as a true ophthalmic subspecialty. Surgical procedures of this type are now among the most commonly performed procedures in medicine today.

The utility of far UV lasers, such as the Ar-F excimer laser, emitting at 193 nm, for large-area surface photoablation on living eye tissue, without any observable dimunition in corneal transparency, was first reported in 1985 (Serdarevic, O. N., et al., “Excimer Laser Therapy for Experimental Candida Keratitis,” Am. J. Ophthalmol. 99: 534-538 (1985)). Since that time, tremendous effort has gone into refining its use in a variety of ophthalmic surgical procedures, including for both refractive vision correction and phototherapeutic revision of the cornea. As a consequence, far-UV laser procedures have gained tremendous momentum. Advances have occurred in parallel, although not always in phase, between both surgical techniques and instrumental technologies, including the increasing use of analytical procedures along with therapeutic procedures to optimize treatment outcomes. Improvement in the overall outcomes of such surgical procedures, defined in terms of both the stable improvement of vision in treated patients, as well as minimization of negative side effects arising from such surgery, has been dramatic. However, even with the advent of alternative surgical procedures designed to address specific shortcomings identified through analysis of the increasing body of patient data, and despite a generally advanced state of knowledge on such essential topics as corneal wound healing and ocular optics, the incidence of negative outcomes remains measurable, although small. Given the increasingly large number of patients undergoing these procedures, even a small percentage of negative outcomes impacts a significant number of patients.

Disclosed herein are methods and compositions designed to optimize the outcomes of refractive vision correction, those outcomes defined by stable, optimal correction of higher and lower order optical aberrations with resulting improved quality of vision, beyond that which is possible with technologies and procedures available in the prior art. Furthermore, the practice of the methods of the present invention should enable expansion of the pool of patients amenable to such procedures to include, among others, those not ideally suited for conventional flap-based techniques, such as, for example, members of Asian races.

Early models of excimer (far-UV) lasers used a broad beam with a diaphragm to create small optical zones in spherical or spherical-cylindrical ablation patterns. More sophisticated lasers emerged using scanning systems or slit beams. Further improvement in laser hardware systems occurred with the development of smaller beam delivery systems associated with eye-trackers. Moreover, more sophisticated algorithms to create smoother aspheric ablations were developed. Custom corneal ablation, in which there is a link between the laser light source and either information from the patient's corneal topography, or from wavefront analysis (measure of total eye aberration), has become a commonplace reality.

Far UV laser vision correction has assumed a position as the most frequently performed procedure for correction of refractive error—the most common type of vision disorder. Excimer laser corneal recontouring is performed for the correction of myopia, hyperopia, astigmatism and presbyopia. Several variants of far ultraviolet laser ablation of the exposed corneal surface, including photorefractive keratectomy (PRK), laser in situ keratomileusis (LASIK), laser-assisted sub-epithelial keratectomy (LASEK), epi-LASIK, and sub-Bowman's layer keratomileusis (SBK) have been developed. These procedures involve laser ablation of the exposed corneal surface under the following conditions: after removal of the corneal epithelium with a laser, chemically, or mechanically (PRK); after chemical lifting of a replaceable epithelial flap (LASEK); a mechanical or laser lifting of a replaceable stromal flap (LASIK); a mechanical lifting of a replaceable “epithelial” flap (that has, in practice, been observed to be a combined epithelial, Bowman's and stromal flap) (Epi-LASIK); or lifting of a replaceable sub-Bowman's layer flap, excised through use of a femtosecond IR laser microkeratome (that, in practice, results in considerable variation among treated patients of the location of the plane of cleavage).

Although early interest in far-UV lasers for use in ophthalmic surgery looked to such lasers as a substitute for steel blades to slice through corneal tissue (a variation of radial keratotomy (RK)), the unique characteristics of the coherent light emitted from a far-UV laser render this light source ideal for accomplishing refractive changes in the cornea via a controlled, shallow surface ablation of wide areas of the visually significant central regions of the corneal surface. Prior to the advent of the use of far-UV light (wavelengths less than 200 nm) in ophthalmic surgery procedures, use of laser light sources in opthalmic surgery had become standard. However, the vast majority of these laser surgical tools utilized light from much lower-energy regions of the electromagnetic spectrum—the infrared (IR), and the visible (VIS) bands. Light of these wavelengths, due to factors such as its lower energy, as well as the typical mechanisms for its delivery, is able to penetrate deeper into the eye and, as a consequence, sees great utility in, for example, retinal surgery. However, use of wavelengths in this region is also characterized by the transmission of considerable energy from the target spot to surrounding tissue, even to the point of causing considerable peripheral tissue damage. In contrast, light from the far-UV region of the spectrum penetrates only a few cell layers into the cornea and causes virtually no damage to tissue surrounding the target area. This is due to the near congruence between the energy of far-UV radiation and the bond energies of the molecules comprising the biochemical components of tissue cells. The energy of the incident UV radiation is of the same order as the relatively high bond strengths of the carbon-hydrogen and carbon-oxygen bonds comprising the biomolecules found in cells. Thus, energy is absorbed with sufficient efficiency, rather than passing through the transparent tissue of the cornea, to break down the chemical bonds holding together the molecules of the cells and ejecting the high-energy molecular fragments caused by such decomposition from the tissue site. This interaction of light energy with tissue leads to an “ablation” (or removal) of the corneal tissue, as that the term “ablation” has come to be used in the field. Thus, far-UV radiation from the excimer laser, interacting to a very shallow depth from the exposed stromal surface, can achieve refractive changes in target areas of the corneal surface with very little risk of damage to surrounding tissues. In contrast, laser surgical procedures involving lower-energy light sources (IR, VIS) interact differently with the tissue and bring about changes in target tissue by very different mechanisms than those involved in far-UV procedures.

The cornea is the outermost layer of the eye and serves as the initial refractive medium through which light interacts with the eye. Unique among all biological tissues is its transparency. In fact, the cornea is a multi-layer construct. Referring to FIG. 1, the outermost (anterior) layer of the cornea is the epithelium. The epithelium, approximately 50-100 μm in thickness (6-7 cellular layers), is composed of non-keratinized squamous cells. The epithelium, in turn, comprises a number of distinct layers, including an outer layer of flattened cells, a layer of polyhedral cells, the basal germinal layer, and a basement membrane (normally in the range of 110-550 nm in thickness) which, in turn, comprises two layers: the lamina lucida, underlying the basal epithelial cell layer, and the lamina densa, proximal to the Bowman's layer. The bare corneal nerve fibers end between the basal cells in the epithelial cell layer, which fact accounts for the extreme sensitivity of the outermost layers of the eye to mechanical abrasion, or trauma of any kind. The basement membrane is in contact with the Bowman's layer, a condensation of the outermost portion of the corneal stroma and, thus, much more similar to the stroma than to the epithelial layer that covers it.

The next layer of the cornea, the stroma, accounts for approximately 90% of the cornea's thickness. It is comprised of elongated bands of Type I and Type V collagen arranged in a lamellar array. These lamellae have an average thickness of 2 μm and extend across the breadth of the cornea. The collagen fibers that make up the lamellae are embedded in a hydrophilic matrix made up primarily of glucosaminoglycan (GAG). Posterior to the stroma is Descemet's membrane, a highly elastic layer that serves as the interface between the stroma and the endothelium. The final, posterior, layer of the cornea is a single cell thick and is referred to as the endothelium.

In photorefractive keratectomy (PRK), the epithelial layer of the cornea is first removed by one of a variety of mechanisms (using either chemical or mechanical means, or light), and subsequent light energy from a far-UV laser is then focused on the exposed corneal surface to achieve refractive corrections. Laser in situ keratomileusis (LASIK) initially was developed to decrease postoperative pain, provide faster visual recovery and create less risk of corneal haze from wound healing than PRK. The principal difference between PRK and LASIK is that in the latter procedure, far-UV radiation impinges on the exposed surface of the cornea at a much lower layer beneath the epithelial surface, in the stroma. To reach this level of penetration, it is necessary to reversibly remove a central portion of the cornea as a flap of tissue, extending down into the stroma, in order to expose a stromal layer to the impinging radiation. This is achieved with either mechanical or laser microkeratomes.

The main advantage advocated for LASIK over PRK is related to maintaining the integrity of the central corneal epithelium. This is believed to lead to increased comfort during the early post-operative period, to allow for more rapid visual recovery, and to potentially reduce the wound healing response, at least that triggered by damage to epithelial cells in the central (flap) region of the epithelium. However, despite maintaining a relatively intact epithelium (except for margins of the flap where the mechanical or photomicrokeratome cuts through the epithelium), the process of creating the stromal flap can trigger a significant wound healing response, as well as lead to other complications with more profound long term consequences for optical outcomes than that associated with PRK. Reduced wound healing, a primary goal for any laser surgery of the cornea, correlates very well with less regression for high corrections and a lower rate of complications such as haze, or any phenomena leading to a reduction in corneal transparency. Thus, any surgical procedure, even if successful in achieving a photoablative revision of the refractive properties of the corneal stroma, cannot be an optimal choice for vision correction unless it also is capable of minimizing the types of cellular responses that are manifest as increases in corneal opacity resulting from factors such as keratocyte activation, stromal fibrosis and epithelial hyperplasia. Such a loss of transparency would lead to a sub-standard optical result for the patient. However, an optimal procedure would achieve the above clinical goal while at the same time avoiding unpredictable alteration of corneal biomechanics and/or alteration of intended laser correction of optical aberrations.

There are fundamental differences in the location and intensity of the wound healing events following PRK and LASIK. For example, after PRK, keratocyte apoptosis (unavoidable in any procedure) and the subsequent events of the healing cascade occur immediately beneath the epithelium and across the entire ablated area. This contrasts with LASIK, in which keratocyte apoptosis takes place at the level of the flap interface (within the stroma), and at the site where the blade penetrated the peripheral epithelium. However, in LASIK, the negative consequences from epithelial damage along the periphery of the flap can outweigh the contribution to these consequences of wound healing responses (such as cellular apoptosis) that occur within the stroma. In addition to cellular apoptosis, there are significant differences (more apparent in earlier instrumental configurations comprising less-refined laser systems) in keratocyte proliferation, and myofibroblast transformation, between PRK for low myopia and PRK for high myopia, and between PRK for high myopia and LASIK for high myopia. In general, higher PRK corrections (those that require deeper ablations/greater removal of corneal tissue to correct higher spherical aberrations) incite more keratocyte apoptosis, keratocyte proliferation and myofibroblast transformation than lower PRK corrections, and these events are less intense in LASIK, even for higher levels of correction for myopia. These observations at the cellular level provide us with an explanation for the differences in clinical outcomes and complications such as haze, that occur after LASIK and PRK, as well as for different levels of correction.

PRK, particularly as a result of advances in laser systems, including improved ablation profiles, is now the better option, compared to LASIK, for mild to moderate wavefront-guided corrections, particularly for cases associated with thin corneas, recurrent erosions, or activities involving a predisposition for trauma (martial arts, military service, contact sports, etc.), creating a particular concern over possible de-attachment of the stromal flap, a concern that can linger for years after surgery.

As LASIK increased in popularity, the frequency of its administration led to a significant compilation of patient data. This wealth of data, in turn, has led to attention on complications relating to creation of the stromal flap, particularly where mechanical defects in such flaps have occurred. Although advances in microkeratome technology have minimized or reduced some of these complications, a number of complication-related conditions have been observed and characterized: LNE—LASIK induced neurotrophic epitheliopathy; DLK—Diffuse lamellar keratitis; lamellar opportunistic infections; and progressive ectasia (keratectasia). Moreover, the creation and manipulation of the stromal flap can lead to inducement of optical aberrations such as coma and spherical aberrations arising from biomechanical modifications to the cornea. Thus, one of ordinary skill in the relevant art would recognize that, due to these considerations, it is desirable to develop surgical procedures that eliminate or significantly reduce the need for stromal flaps, leading to a decrease in the number of surgical complications as well as reducing the magnitude of the unwanted effects, without abandoning many of the advantages recognized as attainable with LASIK.

The desire to eliminate or significantly reduce the occurrence of these complications dictates consideration of alternative procedures utilizing an epithelial flap, such as that disclosed for the invention claimed herein, to reduce these problems and, at the same time, to maintain the safety commonly associated with PRK. Using the corneal epithelium to cover the stroma after laser ablation should theoretically reduce pain and wound healing responses, thereby reducing processes leading to decreased corneal transparency. However currently available methods for disepithelialisation suffer from inherent shortcomings that impose a practical limit on the degree to which it is possible to attain the theoretically available advantages from procedures utilizing an epithelial flap. The main problems are related to epithelial-stromal interactions resulting from damaged basal cells, as well as from incomplete or improper reattachment of the flap where the surgeon has difficulty raising the flap, damage/tearing of the flap during manipulation, drying of the flap, and non-adherence of the flap. However, problems that can occur with the flap such as tearing or non-adherence can result in an outcome (discarding of the damaged flap) that is effectively the same as if the epithelium had been debrided, as in standard PRK.

Several techniques for epithelial removal have been utilized in PRK, including mechanical debridement, laser transepithelial ablation, a rotating brush, and ethanol debridement. All of these techniques are reported to be effective for their immediate purpose. However, a fast and safe method of epithelial removal is essential in order to achieve higher goals defined in terms of optimal surgical outcomes. A smooth, exposed surface to be laser-ablated is believed to be important in obtaining a successful outcome from PRK, or similar procedures utilizing disepithelialisation. Procedures employing reversible removal of an intact epithelial flap or sheet (see below), impose even greater demands on the process of removal of the epithelial layer. To remove the epithelium in a manner that exposes an optimal surface for refractive correction, and at the same time allows for rapid, tight epithelial reattachment and diminishes or eliminates the consequences of triggering avoidable wound healing responses in the stroma or epithelium, by leaving intact the basal epithelial cells and at least one layer of the basement membrane, remains a challenge that has not been met in the prior art.

A surgical procedure effective in producing an epithelial flap that is uniform across the plane of delamination (preferably with minimal introduction of epithelial debris and cytokines into the interface) would be highly advantageous. Furthermore, the location of the plane of delamination within the basement membrane or between the basement membrane and Bowman's layer offers additional advantages. By separating the epithelial layer at the plane of hemidesmosomal attachment (through the basement membrane), an optimally smooth layer is exposed; the basal epithelial layer maintains optimal viability; and reattachment of the epithelial layer is optimized as a result of the strong attachment that occurs in a fairly rapid manner as hemidesmosomal links are reestablished. This would provide both long and short term advantages in comparison to techniques available in the prior art. In the short term, the rapid reestablishment of strong attachments between the epithelial layer and the stroma would reduce pain, prevent exposure of the ablated surface to the tear film and healing epithelium, and enhance the rate of optical recovery. In the long term, particularly for those patients in higher risk fields of life or occupations where physical activity increases the risk of trauma to the surgically-created corneal flap, the improved stability of the reattached epithelial layer is highly valuable. Additionally, an epithelial flap, in contrast to the stromal flap created in LASIK procedures, would leave more stromal tissue available for refractive ablation, minimizing the risk of keratectasia. Also, a cleavage plane through the level of hemidesmosomal linkage provides further advantages in that the basement membrane of the epithelium remains sufficiently intact to retain its barrier/membrane function and, thus, screen the stroma from contact with epithelial cell debris that is known to trigger wound healing mechanisms within the stroma that lead to significant negative side effects such as loss of corneal transparency.

Laser-assisted sub-epithelial keratectomy (LASEK) was developed for the same reasons as LASIK (as an improvement over PRK), but with the added goals of obviating the risks of LASIK-type complications related to creation of the stromal flap. LASEK differs from PRK in the attempted reversible removal of the central portion of the corneal epithelium through attempted application of a dilute ethanol solution (typically 20% aqueous). As in LASIK, the delaminated tissue is replaced on the surface of the cornea after refractive changes in the exposed surface of the cornea are achieved with far-UV laser irradiation.

Dilute ethanol disepithelialisation has been the method of choice in LASEK procedures from its inception, largely due to empirical comparisons to alternative delamination agents such as EDTA, saline, etc. The consensus choice of ethanol was made before it was determined that disepithelialisation occurs within the epithelial basement membrane, leaving the underlying Bowman's layer and stroma essentially intact. Studies have confirmed the very smooth plane of cleavage between the lamina lucida and lamina densa of the basement membrane. In procedures such as LASEK, where the flap is replaced on the treated corneal surface, the condition of the exposed stromal surface, along with the posterior surface of the epithelial flap, is even more critical. The mechanism whereby attachment of the epithelium is achieved through hemidesmosomal links is particularly sensitive to the smoothness of these opposing surfaces. To optimize both the rapidity and the strength (or firmness) of the hemidesmosomal links formed between the exposed stroma and the epithelial flap, it is necessary that both surfaces be optimally prepared. Creation of the epithelial flap alone does not guarantee optimal outcomes to the surgical procedure. In addition, deviations from optimal smoothness can lead to unwanted wound healing responses in the cornea that can lead to negative optical outcomes.

More importantly, as indicated above, if any benefit is to be derived from attempted reattachment of the epithelial layers, the epithelial cells must maintain viability and integrity, particularly basal germinal cells that, if not intact, interact with stromal cells, leading to increased wound healing responses. However, in vitro studies of model systems comprising single cell layers of epithelial cells have indicated that the most common conditions for application of ethanol to the corneal surface for creation of the epithelial flap (18% ethanol for 25 seconds) are sufficient to lead to a toxic effect of the alcohol on epithelial cells such that detrimental wound response mechanisms would result. Thus, it is possible to state that ethanol delamination meets many of the ideal criteria for consistent creation of an epithelial flap. However, this positive result is tempered by recognition that it is impossible to utilize ethanol for disepithelialisation without also experiencing the negative effects arising from ethanol's cytotoxic activity.

The data currently available demonstrate that viability of the epithelium, particularly the basal epithelial layer, is critical for achieving the benefit to be derived from leaving the sheet of epithelium as a protective layer after laser ablation in LASEK. If the concentration of alcohol used is maintained at around 20%, alcohol exposure time remains the most critical factor. Other factors such as the type of alcohol, dilution vehicle (distilled water or balanced salt solution (BSS)), and temperature of the solution contribute to the phenomenon. If the epithelial flap does not have good vitality, the dead cells and cellular debris could provide a mechanical barrier for epithelial healing, as well as proving responsible for negative outcomes in these procedures triggered by wound healing responses. If properly created, however, the epithelial flap in LASEK could have a positive impact on wound healing, inciting a less aggressive response and potentially inciting less haze, provided that cellular responses to ethanol toxicity do not override the advantages resulting from use of an epithelial flap. Indeed, recent data indicate that current methods for removing the epithelium result in loss of epithelial cell viability so that, rather than promoting beneficial healing processes, re-application of the epithelial layer (comprising dead or dying cells) can actually hinder post-surgical recovery when compared to techniques where the epithelial layer is not replaced and regenerates through normal healing processes. This outcome would occur regardless of the skill of the surgeon in creating and manipulating the epithelial flap, or whether or not any mechanical flap complications occurred during surgery.

Advocates of LASEK suggest that, from a short-term perspective, there is less discomfort in the early postoperative period, faster visual recovery, and less haze compared to standard PRK for correction of similar levels of refractive error. In the field, however, there is considerable disagreement over interpretation of much of the accumulated data, particularly with respect to long-term effects where, taken objectively, the data fail to illustrate any significant clinical advantage from LASEK over other surface ablation techniques. In addition, despite the claims of advocates, and the admittedly preferable creation of an epithelial rather than a stromal flap, LASEK must rely on application of a chemical agent, ethanol, that is inherently cytotoxic, even the slightest misuse of which can lead to cell destruction, triggering a cascade of healing responses of the type that are recognized as leading to many of the most common negative effects associated with laser refractive surgery. Despite the potential for LASEK to avoid many of the physiological processes linked to negative surgical outcomes, recent data indicates that no real difference exists in the level of adverse wound healing responses observed among the various surgical techniques for surface ablation.

In an attempt to obviate the need for ethanol in the creation of an epithelial flap, epi-LASIK was developed to separate the corneal epithelium mechanically using a blunt plastic separator on a device with or without an applanator, and operating at low levels of suction. The goal of epi-LASIK included the creation of reproducible, intact sheets of viable epithelium. However, there have been multiple reports in the literature that the epi-LASIK mechanical separation technique separates sometimes through epithelial cells, sometimes through different layers of the basement membrane, and sometimes through Bowman's layer and the stroma. In limited human case studies, both the lamina lucida and lamina densa portions of the basement membrane, as well as the hemidesmosomes, were reported to be intact in many areas. Moreover, experimental and clinical studies with the Pallikaris separator, as well as with other commercially available separators, have revealed “epithelial” flaps containing stroma, Bowman's layer and damaged epithelial cells. This type of inconsistent separation would add the risk of complications related to undesirable and/or unreproducible retention of Bowman's layer and stroma, and very undesirable damage to epithelial cells. Unreliable refractive effect, increased higher order aberrations, and increase haze can also result from epi-LASIK.

In a similar fashion, use of femtosecond IR lasers to remove the epithelium below the Bowman's layer creates additional issues that can interfere with achieving optimal optical results for patients. The majority of these procedures are designed to remove an epithelial layer at approximately 60 μm in thickness. In addition, the greatest level of positional precision reported for these photomicrokeratomes is on the order of 5-10 μm, although, in reality, under conditions of normal operation, precision may be as low as 15-20 μm. Given the accepted degree of variation in epithelial thickness of from 40 to 70 μm, a standard setting with the femtosecond laser of 60 μm will result in considerable variation among patients of the location of the cleavage plane. Nor, given current limitations on spatial resolution in either control of the laser or measurement of thickness of the epithelium, is it likely that these inherent limitations can be adequately addressed. Without more precise location of the plane of cleavage of the epithelial/sub-Bowman's layer, it will be impossible to realize the theoretically available advantages from photodisepithelialisation.

The growing body of data accumulated from laser refractive surgery indicates that the differences in outcome from one technique of surface ablation or “advanced surface ablation” to another are becoming diminishingly small. Likewise, as has been alluded to above, the incidence and magnitude of the complications arising from such techniques have decreased considerably from the earliest years when these procedures were first available. However, the fact remains that as small as the incidence of complications has become, it is still far from negligible and current advances do not seem to be able to provide promise of further reducing this finite level of negative outcomes. Nonetheless, it has become clear that more accurate wavefront-guided laser surgery results are obtained with surface ablation techniques than with LASIK.

This leads to the inevitable question of where do far-UV laser corneal procedures go from here? The current data indicate that in order to optimize favorable outcomes, as indicated by a decrease in optical aberrations resulting from current surgical techniques, it is essential to utilize methods that both take advantage of recent advances in techniques and in technology, and at the same time provide a way in which to avoid the limitations inherent in one or more aspects of the currently available procedures. The directions taken in the art are simply not moving this way. It has even been suggested that complex procedures for in vitro creation of genetically modified epithelial cells for application to disepithelialised corneas following laser ablation could provide a way to address the shortcomings inherent in LASEK-type procedures. It is, therefore, a goal of the present invention to provide methods that allow for reversible removal of the epithelium so as to both maintain its viability and capability for rapid reattachment (and permitting at least one layer of the basement membrane to retain sufficient barrier function to screen the stroma from tear film and epithelial cell debris), and at the same time create an exposed stromal surface that both optimizes laser ablation and promotes successful reattachment, both rapidly and at optimal strength, of the epithelium, while minimizing the potential to trigger adverse wound healing responses. To fully realize this goal, and the potential benefits from significant technical advances in these surgical procedures, it is necessary to change the methods now used to prepare the corneal surface for refractive correction.

### BRIEF DESCRIPTION OF THE FIGURES

FIG. 1 is a section of the human cornea, illustrating the layers of which the cornea is comprised; and

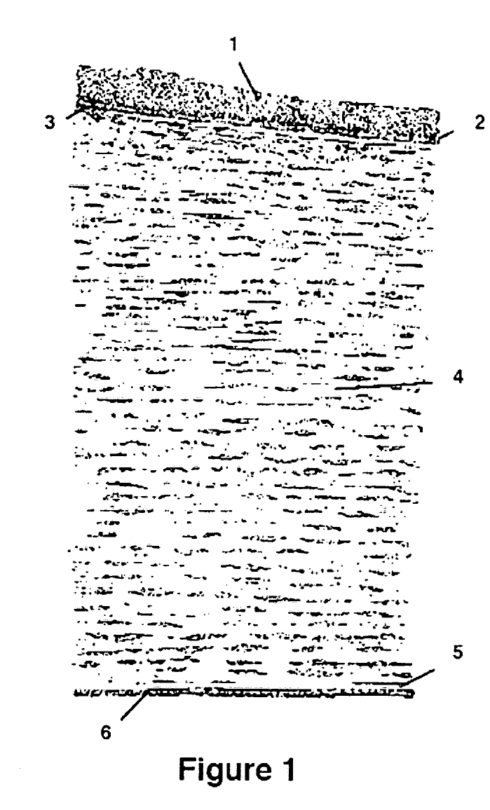

FIG. 2 is representation of the structural components of hemidesmosomes, illustrating the mechanism of attachment.

### DETAILED DESCRIPTION OF THE INVENTION

The method of the present invention is directed to reversible removal of an epithelial flap or sheet from the cornea in such a manner as to create a smooth surface of exposed corneal tissue optimized for both subsequent far-UV laser ablation and rapid, firm attachment of the removed epithelial layer, while at the same time, by maintenance of the basal epithelial cell layer and at least one intact layer of the basement membrane, eliminating or significantly reducing the type of cellular damage that triggers a cascade of biochemical events involved in wound healing that are recognized as contributing directly to some of the most significant complications of laser refractive surgery. A growing body of data from clinical and experimental studies indicates that a critical factor in improving outcomes after laser vision correction is avoidance of basal epithelial cellular and/or tear film interaction with the stroma in order to prevent the triggering of normal cellular “repair” responses in the stroma, which responses are strongly associated with opacification (loss of corneal transparency) and post-operative “haze”. Integrity of the basement membrane can act as a “fibrotic switch” and maintain stromal homeostasis. Thus, a goal associated with the practice of the present invention is avoidance or absolute minimization of disruption of basal epithelial cell membranes through a removal epithelial layer based on attack at binding sites posterior to the basal epithelial cell layer.

The majority of refractive procedures currently performed on the cornea have injury to the epithelium in common. Epithelial injury initiates a sequence of events that occur as part of a protective system for preserving vision. For example, keratocyte apoptosis, the first detectable event after any type of epithelial injury, is associated with either mechanical trauma, corneal surgical procedures, or herpetic (HSV) keratitis, where cellular suicide may provide an early firewall to viral penetration into the eye and central nervous system.

Animal studies have demonstrated that superficial keratocytes undergo programmed cell death mediated by cytokines released from the injured epithelium, such as interleukin (IL)-I alpha, Fas/Fas-ligand, bone morphogenic protein (BMP) 2, BMP4, and tumor necrosis factor (TNF) alpha. Redundancy is probably intended to augment the natural defense system by making it difficult for viral pathogens to overcome a single apoptosis activation system. These cytokines are also present in the tear film, thus making it important to prevent exposure of the treated corneal surface or epithelial cells to the tear film so as to avoid adverse responses triggered by cytokines. Keratocyte apoptosis is followed by a complex cascade of events that takes place in the corneal epithelium and stroma. These events are regulated by cytokine-mediated interactions between epithelial cells, stromal cells, inflammatory cells, nerves, and lacrimal glands. Although some apoptosis cannot be prevented by even the practice of the current invention, the goal of the instant invention is to eliminate, or at least significantly reduce, this cascade of events following apoptosis, and allow for a rapid return to a normal physiologic state of the cornea, with normal regenerative activities rather than repair activities.

Following keratocyte death, the remaining keratocytes surrounding the zone of depletion begin to undergo proliferation within twelve to 24 hours of epithelial injury. At this point, inflammatory cells are also attracted by chemotactic factors such as the monocyte chemotactic and activating factor (MCAF). MCAF production is upregulated in keratocytes by IL-I alpha. IL-I is released from the epithelium after injury, but is also present in the tear film. It appears to be a master modulator of many of the events involved in this cascade. In experiments performed on eyes from patients scheduled to undergo enucleation because of intraocular melanoma, it was confirmed that keratocyte apoptosis and proliferation occur in the human cornea after epithelial scrape (PRK). These events occur in parallel with the closure of the epithelial defect, which is enhanced by growth factors produced by both the lacrimal glands and keratocytes, such as epidermal growth factor (EGF), hepatocyte growth factor (HGF) and keratinocyte growth factor (KGF).

Myofibroblasts are keratocyte-derived cells that are present in the repopulated stromata that are characterized by the expression of alpha smooth muscle actin (SMA). These cells, along with other activated keratocytes, produce disorganized collagen, glycosaminoglycans and growth factors that stimulate healing of the overlying epithelium. Myofibroblasts also have altered transparency in vivo, related to corneal crystallin expression. They are thought to be responsible for, or at least implicated in, the creation of post-operative stromal haze. Differentiation of myofibroblasts is induced by transforming growth factor (TGF) beta, and reversal to fibroblast phenotype has been observed in vitro in the presence of fibroblast growth factor (FGF). TGF-beta, found in the basal layer of the epithelium during its closure, seems to control stromal myofibroblast transformation during corneal repair. In addition, basement membrane formation seems to have an indirect effect on the myofibroblast transformation by regulating the extent of TGF-beta release into the corneal stroma.

There is a return to a normal physiologic state in the corneal stroma several months after injury. This process is associated with eradication of myofibroblasts via programmed cell death or phenotype reversal to quiescent keratocytes. Remodeling of disordered collagen that was produced by myofibroblasts or activated keratocytes during the wound healing process is also mediated by keratocytes. The corneal epithelium may undergo hyperplasia following corneal injury, as a result of the growth factors produced by activated keratocytes and myofibroblasts. Stromal remodeling and epithelial hyperplasia are thought to be the most important mechanisms for regression of the refractive effect of PRK or LASIK surgery.

Immunohistochemical analysis of tissue from the underside of an epithelial flap has shown it to contain the structural elements collagen VII and heparin sulfate, as well as components involved in attachment of the overlying cell to the underlying stroma by hemidesmosomes. These include laminin, fibronectin and entactin-nidogen. Hemidesmosomes are specialized transmembrane cell-matrix junctions between the cytoskeleton of epithelial cells and the extracelleular matrix of basement membranes. The principal component of the hemidesmosomes involved in cell-matrix adhesion is the integrin heterodimer α6β4, a transmembrane protein that can attach to laminin in the basement membrane.

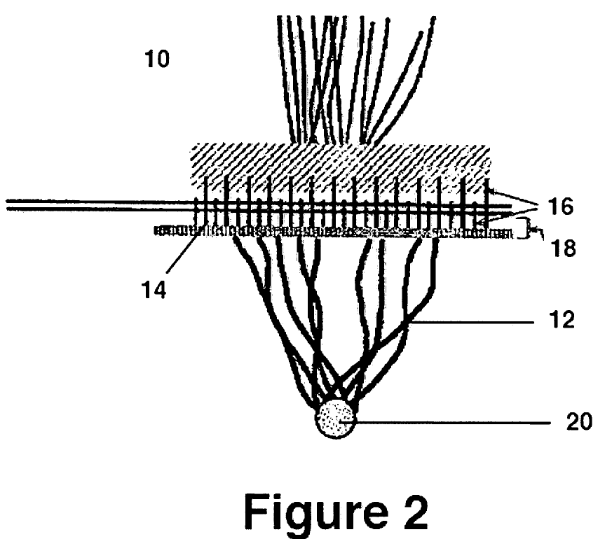

Referring now to FIG. 2, hemidesmosomes (HD) 10 comprise very small stud- or rivet-like structures on the inner basal surface of keratinocytes. Specifically, the HD comprises two rivet-like placques (the inner and the outer placques). Together with anchoring fibrils 12 and anchoring filaments 14, these are collectively referred to as the HD-stable adhesion complex, or HD-anchoring filament complex. The outer plaque contains alpha6-beta4 (α6β4) integrin 16 that functions as the principal structural component involved in adhesion by attaching to laminin 18 in the basement membrane. The anchoring network is composed by anchoring filaments 14 (laminin 5), anchoring fibrils 12 (collagen VII) and anchoring plaque (collagen IV) 20. Together, the HD-anchoring filament complex forms a continuous structural link between the basal keratinocyte filaments and the adjacent basement membrane. Anchoring filaments traverse the lamina lucida and appear to insert into the lamina densa. Beneath the lamina densa, anchoring fibrils can extend within the stroma and physically interact or encircle collagen fibers within the lamellae of the stroma to achieve their anchoring effect between the corneal layers. Thus, the intended cleavage plane of the epithelial flap occurs at the level of anchoring fibrils, either between the epithelium basement membrane and anterior stroma (Bowman's layer), or between the lamina lucida and the lamina densa of the basement membrane.

The HD-mediated interactions between these layers of the cornea are not based on strong covalent links between epithelial cell membrane components and extracellular matrix components. In contrast, the molecular interactions involved are essentially based on much weaker attractions such as hydrogen bonds, Van der Waals interactions, and hydrophobic interactions. These interactions are reinforced by mechanical entanglement of fibrillar macromolecules with cell-membrane receptors and other membrane components. It is important to appreciate that the chemical nature and the energetic magnitude of these interlamellar forces are responsible for both the utility of chemical delamination and the criteria for selection of appropriate delamination agents.

Removal of the corneal epithelium has been found to cause damage to stromal keratocytes, which changes start within 15 to 30 minutes of mechanical disepithelialisation in rabbit and monkey corneas. Other studies have shown that an early decrease in the density of keratocytes is followed by an increased number of these cells in the underlying stroma and production of collagen and extracellular matrix. There is evidence to suggest that keratocyte changes are influenced by the regenerating epithelium (cytokines). Covering of the denuded surface of the cornea directly after a surface-type procedure with the corneal epithelial flap can decrease changes in the stromal keratocytes and minimize the likelihood of the production of extracellular matrix and collagen and the undesirable opacification of the cornea arising from such processes. Although procedures such as LASEK and epi-LASEK involve replacement of an epithelial flap over the photoablated stromal surface, the gain from such a step is severely limited due to the lack of viability of the resulting epithelial cell layer (with disruption of the basal epithelial cells) due to the very process of creating the layer. In the almost inevitable demise of such reattached epithelial layers, these techniques devolve to variations of PRK, with the previously discussed limitations of same.

Demonstrating potentially advantageous diminished wound healing response obtained from LASEK, reduced keratocyte loss was observed after LASEK when compared to PRK in a preliminary rabbit study. This may occur due to a barrier effect from the basement membrane against pro-apoptotic cytokines that are also present in the tear film. For example, the use of a collagen shield diminished keratocyte loss after corneal epithelial scrape in rabbits. Also, lower levels of TGF-beta were detected in the tear film during the first week after LASEK when compared with PRK in the contralateral eye. In addition, a recent study pointed out the importance of the TGF-beta liberated from the healing epithelium for myofibroblast transformation, as well as the effect of the basal membrane as a barrier for this interaction. Thus, it is logical to hypothesize that if an epithelial flap is properly created, less myofibroblast transformation would occur, resulting in less haze.

As recognized in the practice of the present invention, it is important to create an epithelial flap by methods that minimally damage the basal epithelial cells, and that such minimal damage becomes a mere incidental effect of the procedure and not an inherent, and unavoidable, result of the techniques used. Successfully preventing induction of optical aberrations and at least limiting, if not altogether preventing, avoidable repair/wound healing responses after refractive surgery, through use of procedures according to the present invention utilizing epithelial flaps, is a vital goal that cannot now be achieved through procedures of the prior art, even through use of ethanol disepithelialisation as in LASEK procedures.

Moreover, ethanol-assisted epithelial separation has been confirmed to be toxic to epithelial cells in both a dose- and time-dependent manner (see Invest Opthalmol Vis Sci 43: 2593-2602 (2002); and J Cataract Refract Surg 28: 1841-46 (2002)). An increase of only a few seconds beyond the minimal exposure necessary for separation leads to cell death, since ethanol is a solvent of the lipid components of the cellular membrane and causes shrinkage of the cell walls. Ethanol enters the epithelial cells and produces disorganization of the cellular chemistry. Numerous clinical studies have documented that the theoretical goals of repositioning an epithelial flap to facilitate epithelial healing, decrease chemotaxis, reduce inflammation, diminish pain, decrease haze formation, and expedite visual recovery are defeated or at least counteracted due to the effects of ethanol toxicity.

The health and viability of epithelial cells, particularly basal epithelial cells, must be maintained in order to obtain optimal clinical outcomes, including reduction or elimination of the most common side effects of UV laser refractive correction procedures. Before development of the methods of the present invention, it has not been possible to achieve this.

It has proven to be impossible to reproducibly separate an epithelial flap mechanically while, at the same time, maintaining a viable epithelial layer because of the varying thicknesses and curvatures of the cornea at different axes. Delamination at relatively constant thickness would inevitably result in separation of tissue sheets at different levels in different areas of the cornea. Only cryofracture can reproducibly separate the epithelium from the basement membrane, but that laboratory-only process cannot be performed in vivo. Thus, it is an element of the present invention that reproducible creation of a viable, non-damaged epithelial flap can optimally be accomplished pharmacologically, rather than mechanically.

Thus, the method of the present invention is directed to reversible removal of a corneal epithelial flap or sheet in such a manner as to expose both a smooth surface for photoablation and a smooth opposing surface posterior to the epithelial flap, with both surfaces optimized for rapid, strong, stable reattachment of a viable epithelial flap. Under this method, the epithelial cells remain viable with intact cell membranes and intact intracellular junctions. In addition, the basal epithelial cell layer and at least one layer of the basement are optimally preserved intact, diminishing typical wound healing responses, whether such responses are triggered by mechanical damage to epithelial cells or by the cytotoxic effect of chemical agents used to remove the epithelium, or by the tear film. Only in this manner is it possible to maintain epithelial viability which, in turn, can prevent or significantly minimize the most common negative side effects of laser refractive surgery. Furthermore, effective removal of the epithelium should have the added benefit of significantly expanding the pool of patients susceptible to vision correction through laser surgery, including patients with myopia, hyperopia, astigmatism, and presbyopia.

The method of the present invention comprises chemical/pharmacologic separation of the epithelium either from between the basement membrane and Bowman's layer, or from between the lamina lucida and the lamina densa of the basement membrane, leaving: 1.) a very smooth surface to be laser-ablated; and 2.) an epithelial flap with at least one layer of basement membrane and viable, undamaged basal epithelial cells enabling rapid hemidesmosome reformation with firm attachment of the epithelial flap to the underlying surface.

The practices of the prior art have almost exclusively focused on the use of ethanol as a delamination agent. Although this renders it possible to achieve many of the goals of creation of a replaceable epithelial layer, such as a smooth surface for refractive correction, and minimization of damage to the stroma and other elements of corneal anatomy, these achievements do not come without a price. In an empirical fashion, other chemical agents for disepithelialisation have been investigated, but have not demonstrated the same utility in creation of an optimal epithelial flap. Thus, it is an element of the practice of the method of the present invention to utilize chemical agents or compositions selected for a similar activity for attacking the relatively weak, non-covalent interactions through which hemidesmosomes act to bind the layers within the basement membrane, as well as to bind the basement membrane to the underlying Bowman's layer. Chemical/pharmacologic agents may be chosen based on possessing chemical activity similar to ethanol where the agents can interrupt the relatively weak binding forces associated with hydrogen bonding and/or hydrophobic/van der Waal's forces. Of course, these agents must be selected so as to avoid the deleterious effects ascribed to alcoholic agents such as entering the epithelial cell with resulting damage to major cellular components. Relevant among these mechanisms of cytotoxicity is the breakdown of the lipid bi-layer within cells by ethanol, which mechanism is presumed to be correlated to the relatively short carbon chain length of the alcohol. Thus, longer chain-length alcohols, as well as polyhydroxy alcohols, are preferable candidates for the chemical agent of the present invention. At the same time, these agents and/or compositions must be selected on the basis of their relative lack of cytotoxic activity.

By way of example, and without limitation to the scope of the present invention, suitable chemical/pharmacological agents, as would be recognized by one of ordinary skill in the relevant art, could be selected from long-chain, high molecular weight organic solvents displaying milder hydrolytic activities on peptide bonds, along with efficient destabilization of strong molecular hydrophobic interactions. Alternatively, effective epithelial delamination agents could be selected on the basis of an enzymatic approach related to the specific cleavage sites of the fibrillar macromolecules responsible for adhesion of the basement membrane of the epithelium to the Bowman's layer of the stroma, or in combinations of both approaches, in a single or sequentially administered agent or composition.

Suitable delamination agents of the present invention would be selected from long-chain, high molecular weight organic solvents. Such species would display mild hydrolytic activities on peptide bonds, thus acting far less cytotoxic than the current agent of choice for LASEK procedures, ethanol. At the same time, such species would display an ability for efficient destabilization of molecular hydrophobic interactions and/or hydrogen bonds sufficient to counteract the anchoring function of hemidesmosomal complexes. By way of illustration, and without limitation to the scope of the invention disclosed herein, polyhydroxy alcohols and/or polymers of same, meet the necessary chemical criteria for interruption of the binding forces involving HD anchoring between layers of the cornea. Potential cytotoxic effects of such species can be modulated to optimal levels through control/selection of carbon chain length and number of hydroxyl groups on the molecular chain for small molecule species, and molecular weight for polymeric species. Preferably, for polyhydroxy alcohols, optimal carbon chain length would be 4-6 carbon atoms, with 2-3 hydroxyl groups on the carbon chain. For polymeric species, preferred molecular weight ranges would be on the order of 6,000 to 90,000 Da.

A number of compounds useful in the present invention, which have already received FDA approval for ophthalmic use in other applications, include benzyl alcohol, cetyl alcohol, lanolin alcohols, phenethyl alcohol, and polyvinyl alcohol. See CDER Inactive Ingredient Search for Approval Drug Products, a copy of which is incorporated herein by reference in its entirety.

Polyhydroxy alcohols within the scope of the present invention are known in the art. They include ethylene glycol, propylene glycol, glycerol 1,2-propanediol, sorbital, mannitol, inosital, pentaerythritol and the like, the last five being examples of polyhydroxy alcohols having between 4 and 6 carbon atoms. Likewise, polymers of these polyhydroxy alcohols, which also come with the scope of the present invention, are known in the art. They include polyethylene glycol (PEG, such as PEG 300 and PEG 400), polypropylene glycol, polyglycol, polysorbital, polymamitol and the like.

Other alcohols are also suitable for the present invention. These other alcohols include aliphatic alcohols of between three to five carbon atoms in length, including isomers of these alcohols, for example primary, secondary and tertiary alcohols. Thus, alcohols such as n-propyl alcohol, isopropyl alcohol, sec-butyl alcohol, tert-butyl alcohol, n-pentyl alcohol, isopentyl alcohol and other isomers of pentyl alcohol, including cyclopentyl alcohol. Also envisioned are alcohols with six carbon atoms, such as hexanol and cyclohexanol. See N. Kornfield-Paullain, et. al. Effect de Différents Solvants Organiques Sur La Dégredation Alcaline de L' Élastine, 50 Bull. Soc. Chem. Biol. 759-771 (1968), which is incorporated herein by reference in its entirety.

The concentration of the above delaminating agents should be sufficient to cause loosening of the hemidesmosonal links within the cornea. These links function between the anterior epithelial layer of the cornea and the stomal layer of the cornea posterior to the epithelial layer. The concentration should not be so high, however, as to be cytotoxic.

As envisioned, the concentration of the delaminating agent should be from about 0.1% to about 60%. More preferably, the concentration should be from about 5% to about 30%.

In one embodiment, the pH of the delaminating agent is made to be about the same as the pH of the eye, which is about 7.4. In another embodiment, the delaminating agent is isotomic.

### PROPHETIC EXAMPLES

Practice of the method of the present invention, as will be recognized by one of skill on the appropriate art, can be accomplished through procedures adapted from those currently utilized for ethyl alcohol disepithelialisation (LASEK). Accordingly, procedures such as those described below, if utilized with chemical delamination agents selected according to the disclosures and teachings herein, will provide optimal patient outcomes (as defined above) for refractive vision correction with far UV laser radiation.

### Example A

### Chemical Delamination of the Epithelium

Patient is seated comfortably in an appropriate treatment chair. For those patients with a heightened sense of anxiety concerning the impending procedure, pre-administration of approved anti-anxiety medications may be indicated. Typically, a mechanical aid such as a speculum is utilized to allow the treating physician unhindered access to the patient's eye. With or without such an aid, one or more doses of a suitable topical anesthetic are applied the eye to be treated. Preferably, in addition to the topical anesthetic, the eye is also treated with an ophthalmic antibiotic. The regimen of antibiotic therapy may be limited to in situ administration concurrently with pre-operative medications, or it may involve a course of administration begun some days prior to surgery. In addition, non-steroidal anti-inflammatory agents may be used or may be applied topically.

Once the patient and the eye to be treated are prepared, one or more of the chemical agents or pharmacological compositions of the present invention may be applied to the eye. The method or system of application of the delaminating agent do not necessarily comprise a component of the present invention but may rely on techniques and apparatus currently used in similar procedures for laser vision correction. Concentrations of any compositions of the invention, diluent, time of application, method of cessation of application, duration and rigor of rinsing of the treated eye, are all factors, as would be recognized by one of skill in the appropriate art, that would depend on the specific chemical identity of the delaminating agent used. By way of comparison, LASEK procedures, as discussed above, typically utilize solutions of ethanol in a concentration range of 15-20% by volume. At this concentration, exposure times necessary to optimize delamination of the epithelium are in the range of 20-30 seconds. Any longer exposure significantly increases the risk, or likelihood, that the epithelium will experience irreversible cytotoxic damage that could have significant negative impact not only on the continued viability of the epithelium upon reattachment, but also the ultimate outcome of the vision correction procedure. However, one of the advantages of the practice of the method and compositions of the present invention is that the delamination agents are not cytotoxic so that the criticality of time of exposure of the cornea to the agent is no longer an issue. Exposure times are then dictated solely, on one end of the time spectrum, by the exposure duration necessary for the agent to act on the hemidesmosomal links to insure delamination and, on the other end, by consideration of the convenience of the patient and/or treating physician. The present invention also contemplates that the delaminating agents of the present invention will possess a range of efficacies in their delamination function so that exposure times, as well as other parameters of use, will have to be adjusted in accord with choice of specific agent. However, as addressed above, the essential lack of toxicity of these agents removes time of exposure as a critical determinant of the procedural protocol.

At termination of exposure to the delaminating agent, the treated eye is rinsed with an appropriate lavage and/or excess delamination agent may be removed by gentle blotting with a merocel sponge. The lavage could also comprise an NSAID analgesic, such as diclofenac sodium or ketoroloac tromethamine. An alternative, additional step to the procedure would be application of a suitable antibiotic, either before of after treatment with the delaminating agent. At this point in the procedure, the now loosened epithelial flap or sheet is carefully removed and stored or, for procedures involving flap creation, flipped over, for that duration of time necessary for laser treatment of the exposed stroma. The specifics of the devices used and the procedure to be followed are analogous to those used in prior art procedures involving removal of corneal layers, such as epi-LASIK, and do not necessarily comprise an essential component of the practice of the method of the present invention.

The exposed stromal surface of the eye to be treated is now subject to far-UV radiation (preferably from an excimer laser at a wavelength of 193 nm). At this point, either the stored epithelial flap or sheet is returned to the surface of the treated eye and appropriately repositioned with the aid of devices conveniently available, ranging from metallic spatulas or canulas to methylcellulose sponges, or the epithelial flap is repositioned. Optionally, a non-steroidal anti-inflammatory (NSAID) composition may be added to the treated eye. Another option, in place of, or in combination with, NSAID treatment involves administration of an ophthalmologically effective steroid. Finally, a bandage contact lens is applied and the patient is instructed on follow-up care of the treated eye(s).

In a preferred embodiment, the chemical delamination agent is packaged in a pre-portioned, single-dose as part of a kit that may, optionally, contain other compositions or devices with utility in the practice of the present invention. One particularly preferred alternative embodiment comprises a bandage contact lens adapted to function as an aid to removal, temporary storage and re-application of the delaminated epithelial sheet. This embodiment provides particular utility in that the essential bandage contact lens, when used in conjunction with the removal, handling, storage and re-application of the epithelial sheet can significantly reduce the extent of handling or manipulation of the tissue. As would be particularly appreciated by one of skill in the art, any reduction in handling or manipulation of the delaminated epithelial layer will significantly reduce the chances the tissue will suffer damage that would limit, or even prevent, it from providing the advantages of reduction in discomfort and shorter time for visual recovery that can only be realized through reattachment of a viable epithelial layer.

In one embodiment, the bandage contact lens of the invention could be affixed to a mechanical device or surgical tool adapted to draw a slight vacuum through which affixation of the lens to the tool is accomplished or aided. If the bandage contact lens is further adapted in a manner to enhance its porosity, then the vacuum drawn through tool can be applied across the lens so that, when the lens is applied to the loosened epithelial layer on the patient's eye, the suction is sufficient to draw the layer off of the eye and reversibly affix it to the inner surface of the bandage contact lens. In this manner, the mechanical handling of the tissue layer is further reduced providing the benefit of reduced possibility of damage to the delaminated epithelial layer.

These embodiments are provided to aid in illustration of the practice of the present invention only and in no manner are intended, or will serve to, limit in any way the scope of the present invention, which scope is defined in the claims that follow.

I claim:

1. A method for optimizing the outcomes of refractive laser surgery of the cornea, wherein the method comprises the steps of:
a. selecting a delaminating agent effective in loosening hemidesmosomal links within a human cornea of a patient to be treated, wherein the links function between an anterior epithelial layer of the cornea and a stromal layer of the cornea posterior to the epithelial layer; and
b. exposing the cornea to the agent under opthalmologically effective conditions so that the epithelial layer may be reversibly removed from the cornea in a manner permitting continued cellular viability with intact epithelial cell membranes and intact intracellular junctions in the epithelial layer and also permitting rapid, strong, stable reattachment;
wherein the delaminating agent is a primary, secondary or tertiary alcohol of between three and six carbon atoms, cyclopentanol or cyclohexanol.

2. A method of avoiding disruption of basal epithelial cells and at least an anterior layer of a basement membrane in a cornea during laser refractive surgery which comprises applying to the cornea a therapeutically effective amount of a delaminating agent, wherein said delaminating agent is effective in disrupting HD-anchoring complexes so that a uniform cleavage plane is created either between the cornea's lamina lucida and lamina densa or between the cornea's lamina densa and Bowman's layer; and wherein the delaminating agent is a primary, secondary or tertiary alcohol of between three and six carbon atoms, cyclopentanol or cyclohexanol.

3. An improved laser refractive surgery procedure comprising:
a. removing or lifting over an epithelial flap or sheet from an eye;
b. treating an exposed stromal surface of the eye with far-U/V radiation; and
c. replacing or repositioning the exposed epithelial flap or sheet to the eye;
wherein the improvement comprises applying a delaminating agent to the eye prior to removing or flipping the epithelial flap or sheet;
wherein said delaminating agent is effective in loosening hemidesmosomal links within a cornea, wherein the links function between an anterior epithelial layer of the cornea and a stromal layer of the cornea posterior to the epithelial layer; and
wherein the delaminating agent is a primary, secondary or tertiary alcohol of between three and six carbon atoms, cyclopentanol or cyclohexanol.
